(19) United States
(12) Patent Application Publication
(10) Pub. No.: US 2023/0121410 A1
(43) Pub. Date: Apr. 20, 2023

(54) Water-Degradable Environmentally Friendly Sanitary Napkin

(71) Applicant: JI YEUN KIM, Seoul (KR)

(72) Inventor: JI YEUN KIM, Seoul (KR)

(21) Appl. No.: 17/050,189

(22) PCT Filed: Feb. 20, 2020
(86) PCT No.: PCT/KR2020/002460

§ 371 Date: Oct. 23, 2020

(51) Int. Cl. A61F 13/15; A61F 13/56; A61L 15/20; A61F 13/84; A61L 15/46; A61L 15/52; A61L 15/62

(52) U.S. Cl. CPC A61F 13/15211; A61F 13/5611; A61L 15/20; A61F 13/8405; A61F 13/15252; A61L 15/46; A61L 15/52; A61L 15/62; A61F 2013/8414; A61F 2013/8408; A61F 2013/15235; A61F 2013/1556

(57) ABSTRACT

The present disclosure relates to an environmentally friendly sanitary napkin including: a permeable inner sheet configured to come into contact with a skin and allow secretions to pass therethrough; an impermeable waterproof outer sheet having the same size and shape as those of the inner sheet and bonded to the inner sheet by fusion; an absorbent pad fixedly inserted between the inner sheet and the waterproof outer sheet and configured to absorb secretions such as menstrual blood that passes through the inner sheet; and an adhesive pad inserted into an appropriate position between the absorbent pad and the waterproof outer sheet, and provided on the absorbent pad and the lower surface of the waterproof outer sheet, and configured to fix the sanitary napkin by adhesion to the inside of underwear, wherein the inner sheet, the waterproof outer sheet, the absorbent pad and the adhesive pad are degradable in water or are biodegradable.

The environmentally friendly sanitary napkin of the present disclosure is degradable by a large amount of water in a septic tank when flushed down a toilet bowl after use, and is biodegradable in soil, thus preventing environmental pollution problems.

In addition, the environmentally friendly sanitary napkin may exhibit antimicrobial effects against harmful bacteria in the vagina and exhibit an excellent effect on sanitariness by suppressing the generation of odor caused by secretions.

### TECHNICAL FIELD

The present disclosure relates to a water-degradable environmentally friendly sanitary napkin, and more particularly to a water-degradable environmentally friendly sanitary napkin whose constituent components are degradable in a large amount of water or biodegradable in soil, thus preventing environmental pollution problems.

### BACKGROUND ART

In general, women wear sanitary napkins or panty liners to efficiently treat menstrual blood caused by physiological phenomena that occur every month in women. In addition, the sanitary napkin is configured to absorb menstrual blood and keep the genital area clean.

Most of the sanitary napkins have been improved to further enhance the efficiency of absorbing a large amount of secretions by a pad having a small volume or the waterproofing efficiency while minimizing the thickness of a coating agent having fluid impermeability.

The pad described above contains a highly absorbent polymer to increase the ability to absorb secretions, and is characterized by having a great effect on the absorption of secretions due to its high absorption rate despite having a small volume.

Menstrual blood secreted during menstruation in women contains menstrual blood, a cellulosic substance and a mucous substance. This menstrual blood is absorbed by the menstrual blood absorbing pad of the sanitary napkin, but a problem arises in that the absorbed state lasts for a long time and becomes an environment favorable for the growth of various bacteria, thus causing unhygienic conditions or an odor.

Therefore, the hygienic conditions of the sanitary napkin are very important, and the prevention of bacterial growth along with the absorption of menstrual blood and secretions is considered essential for the sanitary napkin.

The following shows an example of a typical sanitary napkin. As described above, the sanitary napkin is used to hygienically treat menstrual blood secreted during the menstrual period. A sanitary napkin that is generally distributed includes: a permeable inner sheet configured to come into contact with a skin and allow secretions to pass therethrough; an impermeable waterproof outer sheet having the same size and shape as those of the inner sheet and bonded to the inner sheet by fusion; an absorbent pad fixedly inserted between the inner sheet and the waterproof outer sheet and configured to absorb secretions such as menstrual blood, which passes through the inner sheet; and an adhesive pad fixedly inserted into an appropriate position between the absorbent pad and the inner sheet and formed at the lower surface of each of the absorbent pad and the waterproof outer sheet, the adhesive pad being configured to fix the sanitary napkin by adhesion to the inside of underwear.

The absorbent pad that absorbs secretions as described above is inserted into an appropriate position between the inner sheet and the waterproof outer sheet, and in this state, the edge of the inner sheet is bonded by thermal fusion to the edge of the waterproof outer sheet. The sanitary napkin configured as described above absorbs, by the absorbent pad, secretions that pass through the inner sheet.

The absorbent pad contains a polymer called superabsorbent polymer (SAP). The superabsorbent resin is a resin that absorbs water tens to hundreds of times its own weight. Since this polymer itself absorbs water, it absorbs a larger amount of water than cotton wool or cotton cloth and does not release water under tolerable pressure. Thus, the polymer is most frequently used for infant, adult or animal diapers. Although the superabsorbent polymer (SAP) has the effect of absorbing water, articles such as diapers and sanitary napkins still have a problem in that they are not self-biodegraded after used for absorption, thus causing environmental pollution.

To solve this problem, there is a need for the development of a sanitary napkin which is soluble in water or biodegradable in soil so as to prevent environmental pollution problems, and also can exhibit an excellent effect on sanitariness by suppressing the generation of odor caused by secretions.

### PRIOR ART DOCUMENTS

### Patent Documents

(Patent Document 1) 10-2016-0022024 A1

### DISCLOSURE

### Technical Problem

An object of the present disclosure is to provide a water-degradable environmentally friendly sanitary napkin.

Another object of the present disclosure is to provide a water-degradable environmentally friendly sanitary napkin which is degradable by a large amount of water in a septic tank when flushed down a toilet bowl after use, thus preventing environmental pollution problems.

Still another object of the present disclosure is to provide a water-degradable environmentally friendly sanitary napkin which is biodegradable in soil when discarded after use, thus preventing environmental pollution problems.

Yet another object of the present disclosure is to provide a water-degradable environmentally friendly sanitary napkin, which may exhibit antimicrobial effects against harmful bacteria in the vagina and exhibit an excellent effect on sanitariness by suppressing the generation of odor caused by secretions.

### Technical Solution

To achieve the above objects, a water-degradable environmentally friendly sanitary napkin according to an embodiment of the present disclosure includes: a permeable inner sheet configured to come into contact with a skin and allow menstrual blood to pass therethrough; an impermeable waterproof outer sheet having the same size and shape as those of the inner sheet and bonded to the inner sheet by fusion; an absorbent pad fixedly inserted between the inner sheet and the waterproof outer sheet and configured to absorb menstrual blood that passes through the inner sheet; and an adhesive pad fixed to the lower surface of the waterproof outer sheet and configured to fix the sanitary napkin by adhesion to the inside of underwear, wherein the inner sheet, the waterproof outer sheet, the absorbent pad and the adhesive pad are disintegrable in water or are biodegradable.

One surface of the waterproof outer sheet is in contact with the absorbent pad, and the adhesive pad is fixed to the other surface of the waterproof outer sheet. The one surface of the waterproof outer sheet, which is in contact with the absorbent pad, prevents menstrual blood, absorbed by the absorbent pad, from passing therethrough, due to the water repellent effect thereof, and the other surface of the waterproof outer sheet, to which the adhesive pad is fixed, easily absorbs water, and thus is disintegrable in water.

The waterproof outer sheet is composed of pulp, and one surface of the waterproof outer sheet may exhibit a water repellent effect due to bonding between the hydroxyl group (—OH) of the pulp and a compound represented by the following Formula 1 or Formula 2:

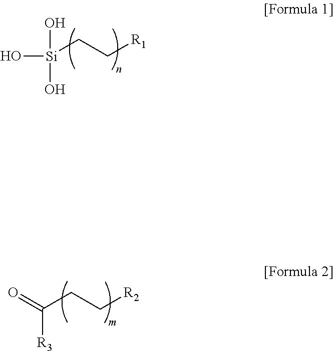

wherein

n and m are the same or different and are each independently an integer ranging from 1 to 10;

R₁ and R₂ are the same or different and are each independently a substituted or unsubstituted C₁-C₁₀ alkyl group;

R₃ is a halogen group; and

R₁ and R₂ are optionally substituted with a halogen group.

The absorbent pad is composed of a plurality of absorbent layers which may be made of a pulp material.

Wing portions may be formed at both lateral sides of the adhesive pad, respectively, and an adhesive layer for adhesion to the inside of underwear may be formed on one surface of each of the wing portions.

The adhesive layer may include a hydrocarbon resin.

The water-dispersible environmentally friendly sanitary napkin of the present disclosure may further include an antimicrobial pad disposed between the inner sheet and the absorbent pad and configured to remove the odor of menstrual blood before the menstrual blood that passes through the inner sheet is absorbed into the absorbent pad.

### Advantageous Effects

The water-dispersible environmentally friendly sanitary napkin of the present disclosure is degradable by a large amount of water in a septic tank when flushed down a toilet bowl after use, and is biodegradable in soil, thus preventing environmental pollution problems.

In addition, the water-degradable environmentally friendly sanitary napkin may exhibit antimicrobial effects against harmful bacteria in the vagina and exhibit an excellent effect on sanitariness by suppressing the generation of odor caused by secretions.

### BRIEF DESCRIPTION OF DRAWINGS

FIG. 1 is a product photograph of a water-dispersible environmentally friendly sanitary napkin according to an embodiment of the present disclosure.

FIG. 2 depicts photographs showing the absorbent effect and waterproof effect of a water-dispersible environmentally friendly sanitary napkin according to an embodiment of the present disclosure.

FIG. 3 shows the results of a water degradability test according to an embodiment of the present disclosure.

FIG. 4 shows the results of a water degradability test according to an embodiment of the present disclosure.

### BEST MODE

The present disclosure is directed to a water-degradable environmentally friendly sanitary napkin including: a permeable inner sheet configured to come into contact with a skin and allow menstrual blood to pass therethrough; an impermeable waterproof outer sheet having the same size and shape as those of the inner sheet and bonded to the inner sheet by fusion; an absorbent pad fixedly inserted between the inner sheet and the waterproof outer sheet and configured to absorb menstrual blood that passes through the inner sheet; and an adhesive pad fixed to the lower surface of the waterproof outer sheet and configured to fix the sanitary napkin by adhesion to the inside of underwear, wherein the inner sheet, the waterproof outer sheet, the absorbent pad and the adhesive pad are disintegrable in water or are biodegradable.

### Mode for Invention

Hereinafter, embodiments of the present disclosure will be described in detail so that those skilled in the art to which the present disclosure pertains can easily carry out the present disclosure. However, the present disclosure may be embodied in a variety of different forms and is not limited to the embodiments described herein.

A water-degradable environmentally friendly sanitary napkin according to an embodiment of the present disclosure includes: a permeable inner sheet configured to come into contact with a skin and allow menstrual blood to pass therethrough; an impermeable waterproof outer sheet having the same size and shape as those of the inner sheet and bonded to the inner sheet by fusion; an absorbent pad fixedly inserted between the inner sheet and the waterproof outer sheet and configured to absorb menstrual blood that passes through the inner sheet; and an adhesive pad fixed to the lower surface of the waterproof outer sheet and configured to fix the sanitary napkin by adhesion to the inside of underwear, wherein the inner sheet, the waterproof outer sheet, the absorbent pad and the adhesive pad are disintegrable in water or are biodegradable.

The inner sheet, waterproof outer sheet, absorbent pad and adhesive pad, which constitute the environmentally friendly sanitary napkin of the present disclosure, are completely degradable in a large amount of water or are biodegradable in soil, and thus are environmentally friendly. In addition, these have an excellent secretion absorption ability, which is the fundamental role of the sanitary napkin, and may exhibit an antimicrobial effect and suppress the generation of odor.

Conventional sanitary napkins have been discarded in enormous amounts, and thus have become the main cause of environmental pollution. In addition, many problems have arisen in that these sanitary napkins generate environmental hormones such as the carcinogenic substance dioxin.

The biggest problem with usual sanitary napkins is the use of chemicals. In the case of commercially available sanitary napkins, polyethylene films, absorbent paper, non-woven fabrics, cotton pulp, and polymer absorbents are indicated as main ingredients, but others are classified as trade secrets and are not disclosed. Disposable sanitary napkins are classified as “infectious wastes” and are buried in the ground after use. In this case, chemicals such as chlorine bleaches permeate the ground and rivers and cause environmental pollution.

The average woman has about 500 menstrual periods for 37 years from 13 to 50 years old, and uses about 11,000 sanitary napkins all her life on the assumption that she uses an average of five sanitary napkins a day for 5 days.

Disposable sanitary napkins are easily used and discarded, but the amount thereof is so great that 2 billion disposable sanitary napkins are discarded in one year and reach 20,000 km when lined up.

In addition, it is known that the production of 11,000 sanitary napkins is possible only when a forest corresponding to the size of Yeouido is destroyed every year.

As mentioned above, disposable sanitary napkins not only destroy the environment, but also adversely affect women's health. In recent years, problems have arisen in that bugs are found in some sanitary napkin products or formaldehyde is detected in some products.

Against these problems, methods of using alternative sanitary napkins have recently been discussed, and it is known that these methods are broadly divided into the use of pad-type cotton sanitary napkins and the use of insertion-type alternative sanitary napkins.

The pad-type cotton sanitary napkin is a sanitary napkin which is made of cloth so as to be washable and reusable continuously. This cotton sanitary napkin has less skin irritation and is considered an environmentally friendly product that is reusable multiple times, but has the problem of being difficult to wash after use.

In addition, as the insertion-type alternative sanitary napkin, two types are commercially available: Keeper made of natural rubber, and Moon Cup made of silicone. The insertion-type alternative sanitary napkin is reusable multiple times, like the cotton sanitary napkin, but needs to be stored in a cool place due to the nature of the material, and has the problem of being difficult to wash.

Accordingly, the present disclosure is intended to provide a sanitary napkin that has no significant difference in the configuration from a conventional sanitary napkin, but is composed of constituent components that are all degradable in water or biodegradable in soil, thus preventing the occurrence of environmental problems.

The water-degradable environmentally friendly sanitary napkin according to an embodiment of the present disclosure includes an inner sheet, a waterproof outer sheet, an absorbent pad and an adhesive pad. The inner sheet, the waterproof outer sheet, the absorbent pad and the adhesive pad are composed of pulp, and may be easily disposed of by putting the sanitary napkin in a toilet bowl or landfilling the sanitary napkin.

That is, when the sanitary napkin is put in a toilet bowl and the toilet bowl is flushed with water, it is moved to a septic tank by water pressure, and then moved to a sewage treatment plant and treated therein.

When the water-degradable environmentally friendly sanitary napkin of the present disclosure is put in a toilet bowl after use and the toilet bowl is flushed with water, a phenomenon occurs in which the inner sheet, the waterproof outer sheet, the absorbent pad and the adhesive pad are disintegrable due to degradation action caused by water absorption upon contact with water. Thereafter, when the sanitary napkin is moved to a septic tank by water pressure, it may be completely degraded in the septic tank so that it may be easily purified in a sewage treatment plant.

In addition, as the phenomenon occurs in which the shape of the sanitary napkin disintegrates by water absorption upon contact with water and completely disintegrates by water pressure, flushing the sanitary napkin down a toilet bowl does not cause the problem that the toilet bowl is clogged.

In general, a large amount of tissues are flushed down the toilet bowl or when an item that is insoluble in water is flushed down the toilet bowl, a phenomenon occurs in which a drain pipe connecting the toilet bowl to a septic tank becomes clogged.

If the drain pipe is clogged, when using the toilet, a problem arises in that a backflow phenomenon occurs when the toilet bowl is flushed with water.

Most recent toilet tissues are easily disintegrable when flushed down toilet bowls, and thus do not cause the problem that the drain pipe is clogged. This is because the shape of the toilet tissue easily disintegrates by strong water pressure when the toilet tissue is moved to the drain pipe, and thus does not cause the problem that the drain pipe is clogged. In addition, the toilet tissue is easily disintegrable in a large amount of water by virtue of the improved quality thereof, and thus the toilet clogging problem is overcome.

Toilet tissue is composed of pulp that easily absorbs water, and thus a disintegration phenomenon easily occurs in which the shape of the toilet tissue disintegrates by water absorption.

Generally, products such as toilet tissue made of pulp may easily absorb water, but when these products are specially processed, they may exhibit water repellent performance, and thus may also be manufactured so that they do not easily disintegrate in water

The inner sheet, waterproof outer sheet, absorbent pad and adhesive pad, which constitute the water-dispersible environmentally friendly sanitary napkin of the present disclosure, may all be composed of pulp so that they may easily absorb water and easily disintegrate in a large amount of water, thus exhibiting water degradability.

Pulp has the property of easily absorbing water. Thus, when pulp is used for a sanitary napkin, it may easily absorb menstrual blood and retain the absorbed menstrual blood therein.

In addition, even when the sanitary napkin is flushed down a toilet bowl, it will be in contact with a large amount of water and water pressure will act directly on the sanitary napkin, so that the shape of the sanitary napkin may easily disintegrate.

It is important for a usual sanitary napkin to easily absorb menstrual blood and to exhibit the performance of retaining the absorbed menstrual blood without leakage to the outside.

That is, in order to prevent the absorbed menstrual blood from leaking to the outside, the absorbent pad includes a polymer absorbent material and the waterproof outer sheet is made of a plastic material such as vinyl resin, which has an excellent waterproof property, thus preventing menstrual blood from leaking to the outside.

However, as described above, when a polymer absorbent material and a polyethylene film are used as main components, as in a conventional sanitary napkin, the sanitary napkin may cause environmental pollution problems when discarded after use, and may also easily cause problems such as bacterial growth, vaginitis and dermatitis, due to the poor air permeability thereof during use.

According to the present disclosure, these problems may be easily prevented by using a pulp material having air permeability.

In addition, the absorbent pad is composed of a plurality of absorbent layers made of a pulp material so that it can sufficiently retain even a large amount of menstrual blood.

In addition, one of the absorbent layers may be configured to have a density of 80 to 120 g/m², and the absorbent pad may be composed of 5 to 10 absorbent layers. However, the present disclosure is not limited to the above example, and the number of absorbent layers in the absorbent pad may be easily selected within a range predictable by those skilled in the art so that the absorbent pad may be used to retain menstrual blood.

As described above, the absorbent pad corresponding to the most important component for the sanitary napkin does not include a separate polymer absorbent material, and is composed only of absorbent layers made of a pulp material. Due to the characteristic air permeability of the pulp, it is possible to prevent bacterial propagation, vaginitis or dermatitis from developing when the sanitary napkin is used.

In addition, as the absorbent pad is composed of a plurality of highly dense absorbent layers, it may sufficiently retain menstrual blood regardless of the amount of menstrual blood, indicating that the absorbent pad may sufficiently absorb menstrual blood.

The permeable inner sheet is a surface that comes into direct contact with the human skin during the use of the sanitary napkin, and serves to prevent skin irritation, allow menstrual blood menstrual blood to pass therethrough, and allow menstrual blood to be transferred to the absorbent pad.

The inner sheet of the present disclosure is composed of a pulp material and configured to have a density of 30 to 40 g/m². Thus, the inner sheet is composed of a pulp material having excellent permeability and processed to have a soft skin contact surface to prevent skin irritation.

If a pulp material having a density of less than 30 g/m² is used to increase the permeability of the inner sheet, a problem may arise in that the inner sheet has weak durability, and thus is easily damaged by an external force when a woman wearing the sanitary napkin sits on a chair or performs strenuous exercise.

To prevent this problem, the inner sheet is composed of a pulp material having a density of 30 to 40 g/m², so that the durability problem may be solved, and at the same time, skin irritation may be lowered and menstrual blood may be easily absorbed into the absorbent pad.

The sanitary napkin of the present disclosure may further include, between the inner sheet and the absorbent pad, an antimicrobial pad capable of removing the odor of menstrual blood before the menstrual blood that passes through the inner sheet is absorbed into the absorbent pad.

The antimicrobial pad is configured such that the menstrual blood that passes through the inner sheet is easily movable to the absorbent pad. Thus, it is possible to easily remove the odor of menstrual blood through the antimicrobial pad, and at the same time, it is possible to prevent an infection problem from being caused by harmful bacteria in vagina since the absorbent pad retains the menstrual blood.

The antimicrobial pad may be composed of a pulp material, and both surfaces thereof may be coated with an antimicrobial composition containing a natural extract.

The sanitary napkin of the present disclosure is characterized by including no chemical synthetic product in order to provide an environmentally friendly sanitary napkin. Thus, the antimicrobial pad also contains a natural extract in order to increase the antimicrobial effect and odor removal effect thereof.

The antimicrobial composition may contain one selected from the group consisting of a Diospyros kaki leaf extract, a Citri pericarpium extract, a Salicornia herbacea extract, a banana peel extract, and mixtures thereof. Preferably, the antimicrobial composition may contain, as an active ingredient, an extract mixture of a Diospyros kaki leaf extract, a Citri pericarpium extract, a Salicornia herbacea extract and a banana peel extract.

Diospyros kaki Thunb is mainly distributed in the central and southern regions of Korea. In addition to Diospyros kaki fruits that are eaten raw, Diospyros kaki leaves, flowers and bodies have been used as folk remedies from ancient times. Among them, in particular, Diospyros kaki leaves have been mainly used as tea, and it is recorded in Donguibogam (Korean medical encyclopedia written by Heo Jun) that Diospyros kaki leaves have effects on the prevention and treatment of vascular diseases and are also effective against pelioma (vascular inflammation). Diospyros kaki leaves contain various substances such as vitamin C, amino acids, nucleic acids, phenolic compounds, free sugars, etc. Flavonoids in Diospyros kaki leaves include glucoside forms such as astragalin (a glucoside of kaempferol), myricitrin (a glucoside of myricetin), and isoquercitrin. Diospyros kaki leaves have a total phenolic compound content of 0.1 to 5.8%, which is higher than those of other plants, and these leaves have been found to have excellent antioxidant and antimicrobial activities and are known to be involved in superoxide dismutase (SOD) activity. In addition, Diospyros kaki leaves have been proven to have pharmacological effects on the maintenance of skin elasticity and the prevention of colds and adult diseases, strongly suggesting the possibility that various substances in Diospyros kaki leaves can influence human physiological mechanisms. Phenolic compounds have been reported to have antioxidant, antimicrobial and anti-allergic activities and to be effective in the prevention of tooth decay, heart disease and diabetes, and include flavonoids, phenolic acid, phenylpropanoids, quinones, and the like.

Citri pericarpium is one obtained by drying the peel of a mature fruit of Citrus unshiu Markovich which is an evergreen small tree belonging to the family Orchidaceae. In the Korean Pharmacopoeia, it is known that the content of hesperidin (which is a major flavonoid component) in Citri pericarpium is 4% or more, and naringin, neohesperidin, and the like are also contained in Citri pericarpium. Citri pericarpium is used as an aromatic stomach-strengthening drug in gastroenteritis, indigestion, etc. and is also used as an antiemeticum, an antitussive, and an expectorant.

Salicornia herbacea grows in a cluster around the seashores, mud flats or salt farms where seawater reaches, island regions of Korea, such as the west coast, the south coast, Jeju Island, Ulleung Island, and Baengnyeong Island. Salicornia herbacea is called ‘Toongtoongmadi’ in Korean language because it is a grass which is bumpy and has protruding nodes. In Shen Nong Ben Cao Jing which is the old medical book of China, Salicornia herbacea was called Hamcho (salt-containing grass) or Yeomcho (salt glass) since it was very salty in taste, and was also Shincho since it was regarded as a very rare and sacred grass. It has been known since the time of China's Zhou Dynasty (3,000 years ago) that Salicornia herbacea has excellent efficacy as a health food. It is recorded in Rites of Zhou that the king of the Zhou Dynasty served Salicornia herbacea to the heavens and performed rituals. In Daehwaboncho written by Kaibara who is the father of Japanese medicine, Salicornia herbacea is written as a precious grass that allows eternal youth, and in addition to Hamcho, several names such as Yeomcho, Bokcho, and Samji are written. The general components of Salicornia herbacea mainly consist of carbohydrates, ash, crude proteins, crude fats, and salt accounts for 13.5 wt% based on the weight of ash. In addition, it is known that Salicornia herbacea contains various minerals, that is, 100 g of Salicornia herbacea contains 670 mg of calcium and 70 mg of iodine, and has a sodium content of about 6.5%, a salt content of about 16% and a vegetable fiber content of about 50%.

Banana is a perennial plant native to tropical and subtropical regions of Asia. In Taiwan, bananas are mainly planted in Kaohsiung, Tainan, Pingdong, and Taitung. In general, the edible part of the banana is the flesh part and the peel thereof is discarded.

Regarding the production of the natural extract, the natural extract may be obtained by a method including steps of: grinding a natural product; leaching the ground product with a solvent; drying the sample after leaching; leaching the dried sample again with a solvent; drying the sample after leaching; leaching the sample with water; and leaching the sample.

The method may further include a step of subjecting the natural extract, obtained by extraction with the solvent, fractionation with a solvent

The method for producing the extract may be a conventional extraction method known in the art, such as an ultrasonic extraction method, a leaching method, or a reflux extraction method. Specifically, the extract may be an extract obtained by extracting a natural product, from which impurities have been removed by washing and drying, with water, an alcohol having 1 to 6 carbon atoms, or a mixed solvent thereof, or may be an extract obtained by sequentially applying the solvents to the sample.

The extraction solvent may be used in an amount 2 to 50 times, more specifically 2 to 20 times, the weight of the sample. The sample to be extracted may be left to stand in the extraction solvent for leaching for 1 to 72 hours, more specifically 24 to 48 hours.

After extraction, the extract may be fractionated by sequentially applying fresh fractionation solvents. The fractionation solvents that are used for fractionation may be selected from the group consisting of water, hexane, butanol, ethyl acetic acid, ethyl acetate, methylene chloride, and mixtures thereof.

After the extract or fraction is obtained, a process such as concentration or freeze-drying may additionally be performed.

Preferably, the Diospyros kaki leaf extract, the Citri pericarpium extract, the Salicornia herbacea extract and the banana peel extract may be a hot-water extract of Diospyros kaki leaf, a hot-water extract of Citri pericarpium, a hot-water extract of Salicornia herbacea, and a hot-water extract of banana peel, respectively.

Water is used as the extraction solvent without using an organic solvent such as an alcohol, which is commonly used in the production of natural extracts, in order to prevent the possibility that skin irritation occurs due to the natural extract obtained using the organic solvent.

Preferably, the antimicrobial composition may contain, based on 100 parts by weight of the Diospyros kaki leaf extract, 50 to 100 parts by weight of the Citri pericarpium extract, 10 to 20 parts by weight of the Salicornia herbacea extract, and 10 to 20 parts by weight of the banana peel extract. When the extracts are used in amounts within the above-described ranges, they may exhibit an excellent effect of removing the odor of menstrual blood, together with an antimicrobial effect.

More preferably, the antimicrobial composition may be formed into an antimicrobial layer by mixing the Diospyros kaki leaf extract, the Citri pericarpium extract, the Salicornia herbacea extract and the banana peel extract, which are in powder form, in water as a solvent, spraying the mixture onto the antimicrobial pad, and drying the sprayed mixture.

The method of forming the antimicrobial layer is not limited to the spraying method, and a person skilled in the art may select a method of forming the antimicrobial layer using the antimicrobial composition and may use the selected method without limitation.

The water-degradable environmentally friendly sanitary napkin of the present disclosure includes an impermeable waterproof outer sheet having the same size and shape as those of the inner sheet and bonded to the inner sheet by fusion. One surface of the waterproof outer sheet is in contact with the absorbent pad, and an adhesive pad is fixed to the other surface thereof.

The one surface of the waterproof outer sheet, which is in contact with the absorbent pad, prevents menstrual blood, absorbed by the absorbent pad, from passing therethrough, due to the water repellent effect thereof, and the other surface of the waterproof outer sheet, to which the adhesive pad is fixed, easily absorbs water, and thus is disintegrable in water.

That is, the one surface of the waterproof outer sheet, which is in contact with the absorbent pad, may prevent menstrual blood, which passes through the absorbent pad, from leaking to the outside, due to the water repellent effect thereof.

On the other hand, the other surface of the waterproof outer sheet is a surface which is comes into direct contact with water when the used sanitary napkin is flushed down a toilet bowl. The other surface may easily absorb water, causing disintegration.

In the case of a conventional sanitary napkin, the absorbent pad is a polymer film whose both surfaces all exhibit a water repellent effect, thus preventing the problem that menstrual blood absorbed through the absorbent pad from leaking to the outside.

That is, leakage of menstrual blood out of the absorbent pad may be primarily prevented by maximally absorbing menstrual blood using the absorbent material in the absorbent pad, and may be secondarily prevented using a waterproof outer sheet composed of a polymer film.

However, when the absorbent material or the polymer film is used as described above, it is possible to prevent menstrual blood from leaking to the outside during the use of the sanitary napkin, but various problems may arise as mentioned above.

Accordingly, in the present disclosure, a water-repellent coating layer may be formed on one surface of a pulp material by bonding a compound represented by the following Formula 1 or Formula 2 to the surface:

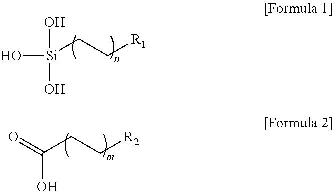

wherein

n and m are the same or different and are each independently an integer ranging from 1 to 10;

R₁ and R₂ are the same or different and are each independently a substituted or unsubstituted C₁-C₁₀ alkyl group;

R₃ is a halogen group; and

R₁ and R₂ are optionally substituted with a halogen group.

In general, a pulp material has hydroxyl groups (—OH) formed on the surface thereof. Through these hydroxyl groups, the pulp material may exhibit a hydrophilic property and easily absorb water.

In the present disclosure, based on this property, the compound represented by Formula 1 or Formula 2 may be bonded to the hydroxyl groups on the pulp surface so that the pulp surface may exhibit a hydrophobic property, thus exhibiting a water-repellent effect.

The compound of Formula 1 contains three hydroxyl groups in silane, and thus the hydroxyl groups may be bonded via hydrogen bonds to the hydroxyl groups on the pulp surface.

In addition, in the case of the compound of Formula 2, R₃ is a halogen group that may form a bond with the hydroxyl group on the pulp surface by nucleophilic substitution.

R₁ and R₂ in Formula 1 or Formula 2 are the same or different C₁-C₁₀ alkyl groups which may be substituted with a halogen group. In particular, when R₁ and R₂ are substituted with fluorine as a halogen group, the water-repellent effect is further increased.

The compound of Formula 1 or Formula 2 is sprayed onto one surface of pulp and heated in an oven, thereby forming bonds with the hydroxyl groups on the pulp.

In addition to the method of heating in the oven after spraying, any method capable of forming a water-repellent layer may also be used without limitation.

The absorbent pad may have a monolayer or multilayer structure formed of the above-described material in which the water-repellent layer is formed on pulp. In addition, if necessary, the absorbent pad may be configured to include a polymer film layer.

That is, the absorbent pad may include a pulp material having a water-repellent layer formed on one surface thereof, and may further include a water-repellent film layer.

The sanitary napkin may be produced in consideration of a condition in which the sanitary napkin is used. When the sanitary napkin is used for a long time or used on days with a lot of menstrual blood, the use of the absorbent pad only composed of a pulp material may cause a problem in that menstrual blood leaks out of the sanitary napkin.

To prevent this problem, a water-repellent film may further be included to prevent menstrual blood from leaking to the outside.

As the water-repellent film, any known conventional product may be used without being limited to one example, and any product selectable by those skilled in the art may be used.

In addition, the water-repellent film may be configured to have a thickness of 1 to 2 mm, so that, when the sanitary napkin is flushed down a toilet bowl after use, the water-repellent napkin is easily degradable by water pressure. However, the thickness of the water-repellent film is not limited to the above range, and any water-repellent film that is applicable to a sanitary napkin by a person skilled in the art may be used without being limited to the above example, as long as the material of the water-repellent film is easily degradable in a large amount of water.

Wing portions are formed at both lateral sides of the adhesive pad, respectively, and an adhesive layer for adhesion to the inside of underwear is formed on one surface of each of the wing portions.

The adhesive layer may include a hydrocarbon resin. In addition to the resin, any adhesive material may be used without being limited to the above example.

### PREPARATION EXAMPLE 1

Preparation of Water-Degradable Environmentally Friendly Sanitary Napkin

1. Preparation of Waterproof Outer Sheet

As a reagent, a compound represented by the following Formula 3 was evenly sprayed onto one surface of a pulp material having a density of 50 to 60 g/m², and was heated in an oven at a preheated temperature of 80 to 100° C. for about 3 to 5 seconds, thereby preparing a waterproof outer sheet (AB1):

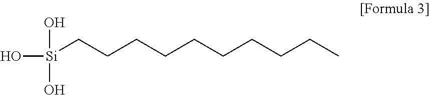

A waterproof outer sheet (AB2) was prepared in the same manner as described above, except that a compound of the following Formula 4 was used instead of the compound of Formula 3.

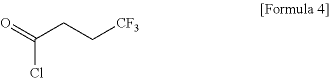

In a comparative example, a pulp material having a density of 50 to 60 g/m² was used as a waterproof outer sheet.

2. Preparation of Antimicrobial Pad

(1) Preparation of Natural Extract

Diospyros kaki leaves were washed clean, dried, and then ground using a grinder. Next, 50 g of the ground Diospyros kaki leaves were added to 500 ml of water at 90 to 100° C. and extracted under stirring for 3 hours. The extract was filtered, concentrated under reduced pressure using a rotary vacuum evaporator (Eyela N-1N), and then freeze-dried using a freeze dryer (FD SFDSM12, Samwon, Korea), thereby preparing freeze-dried Diospyros kaki leaf powder.

From Citri pericarpium, Salicornia herbacea and banana peel, the respective extracts were prepared in the same manner as the Diospyros kaki leaf extract.

(2) Preparation of Antimicrobial Composition

The Diospyros kaki leaf extract, the Citri pericarpium extract, the Sahcornia herbacea extract and the banana peel extract were mixed together in amounts within the ranges shown in Table 1 below, thereby preparing extract mixtures. Each of the extract mixtures was diluted with a 10-fold volume of water, thereby preparing antimicrobial compositions.

TABLE 1
(unit: parts by weight)

| | AR1 | AR2 | AR3 | AR4 |
|---|---|---|---|---|
| Diospyros kaki | 100 | 100 | 100 | 100 |
| Citri pericarpium | 30 | 50 | 70 | 100 |
| Salicornia | 5 | 10 | 15 | 20 |
| herbacea | | | | |
| Banana peel | 5 | 10 | 15 | 20 |

(3) Preparation of Antimicrobial Pad

Each of the antimicrobial compositions (AR1 to AR4) was sprayed onto a pulp material having a density of 30 to 40 g/m² and dried, thereby preparing antimicrobial pads.

3. Preparation of Water-Degradable Environmentally Friendly Sanitary Napkin

An inner sheet was formed from a pulp material having a density of 30 to 40 g/m², and an absorbent pad was prepared by stacking 5 to 10 absorbent layers, each having a density of 80 to 120 g/m².

The waterproof outer sheets coated with AB1 and AB2, respectively, were formed in a sanitary napkin shape.

The antimicrobial pads coated with AR1 to AR4, respectively, were also formed in a sanitary napkin shape.

An adhesive pad was prepared from a pulp material having a density of 80 to 120 g/m².

The inner sheet, the antimicrobial pad, the absorbent pad, the waterproof outer sheet and the adhesive pad were sequentially stacked together, and the inner sheet and the adhesive pad were bonded to each other using an adhesive. Thereafter, an adhesive layer composed of a hydrocarbon resin was formed was formed on the wing portion of the adhesive pad.

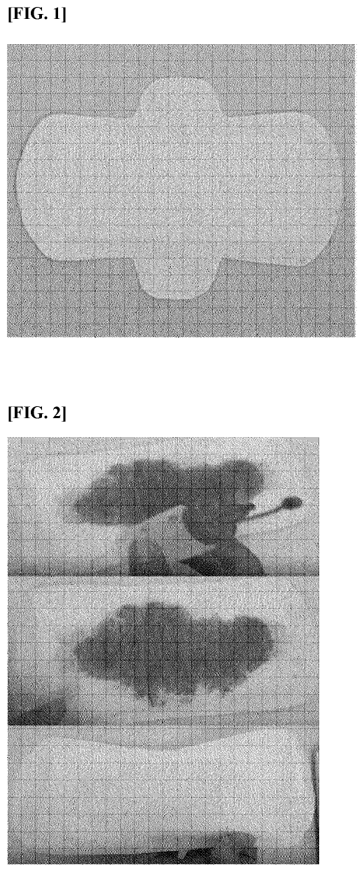

The sanitary napkin according to an embodiment of the present disclosure was prepared in the shape shown in FIG. 1. The shape of the sanitary napkin is not limited to the above-described shape, and the sanitary napkin may be prepared in the shape of any commercially available sanitary napkin.

### EXPERIMENTAL EXAMPLE 1

Mensural Blood Absorption Effect and Waterproof Effect

To evaluate the menstrual blood absorption effect and the waterproof effect, about 50 ml of water containing red ink diluted therein was prepared. The amount of menstrual blood during one menstrual cycle in the average woman is about 80 ml, or 100 ml to 150 ml at most.

In consideration of a condition in which the sanitary napkin is used, the amount of menstrual blood within the replacement period was predicted to be about 30 ml, and whether the sanitary napkin can absorb a larger amount of water than the corresponding amount and the waterproof effect of the sanitary napkin were examined.

As shown in FIG. 2, water was sprayed onto the sanitary napkin and whether the water was sufficiently absorbed and whether the water leaked to the opposite side of the sanitary napkin were examined.

After the absorbent pads were all prepared to have ten absorbent layers in the same manner and only the waterproof outer sheets were changed into AB1, AB2 and the comparative example, the experiment was performed.

When water was absorbed well as shown in FIG. 2, absorption was marked as 0, and when water was not absorbed, absorption was marked as X.

In addition, when red water was found on the opposite side, waterproof was marked as O, and when water did not leak to the outside as shown in FIG. 2, waterproof was marked as X.

TABLE 2

| | | | Comparative |
|---|---|---|---|
| | AB1 | AB2 | example |
| Absorption | ◯ | ◯ | ◯ |
| Waterproof | X | X | ◯ |

As a result of the experiment, it was confirmed that, when the absorbent pads were all kept the same, the absorption effect was the same between AB1, AB2 and the comparative example, but the waterproof effect did differ depending on whether the water-repellent layer was formed. In the case of AR3 in which the water-repellent layer was not formed, it was confirmed that the red ink leaked to the outside and the hand was stained with the red ink, and in the case of AR1 and AR2, this ink stain phenomenon did not occur.

### EXPERIMENTAL EXAMPLE 2

### Examination of Water Disintegration Phenomenon

In order to confirm the phenomenon in which the sanitary napkin disintegrates in water, an experiment was performed. As the waterproof outer sheet, each of AB1 and AB2 of the present disclosure and the comparative example was used.

To examine the degree to which the sanitary napkin disintegrates in water, the water pressure in a toilet bowl was considered, and whether the sanitary napkin disintegrated in water when not stirred and whether the sanitary napkin disintegrated in water when stirred with a spoon were examined.

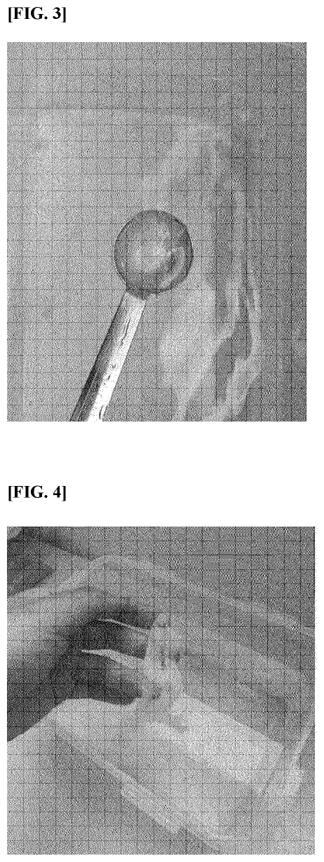

FIG. 3 shows an example in which disintegration in water occurred, and FIG. 4 shows an example in which disintegration in water did not occur.

TABLE 3

| | | | Comparative |
|---|---|---|---|
| | AB1 | AB2 | example |
| Left to stand in water | Δ | Δ | Δ |
| Stirred in water | ◯ | ◯ | ◯ |

According to the above experimental results, it was confirmed that, in all the cases in which each of AB1, AB2 and the comparative example was used as the waterproof outer sheet, all the sanitary napkins of the present disclosure disintegrated. However, in the case of the absorbent pad, it was conformed that, when the absorbent pad was simply left to stand in water as shown in FIG. 4, complete disintegration did not occur, but when the absorbent pad was lightly stirred with a spoon, disintegration occurred.

### EXPERIMENTAL EXAMPLE 3

### Experiment on Antimicrobial Effect

For evaluation of antimicrobial activity, sanitary napkins prepared under the same conditions, except only the antimicrobial pad, were inoculated with a fungus, and after 24 hours, the concentration of the fungus was measured. The results of the measurement are shown in Table 2 below.

The fungus is known to cause Candida vaginitis. An experiment on the antimicrobial effect against the fungus was performed.

TABLE 4

| | | | Microbial |
|---|---|---|---|
| | Initial | Log | reduction |
| | concentration | (CFU/mL) | rate |
| | (CFU/mL) | after 6 hours | (%) |
| Comparative | 1.00 × 10⁵ | 8.00 × 10⁵ | — |
| Example | | | |
| AR1 | 1.00 × 10⁵ | 8.00 × 10⁵ | — |
| AR2 | 1.00 × 10⁵ | 0 | 100 |
| AR3 | 1.00 × 10⁵ | 0 | 100 |
| AR4 | 1.00 × 10⁵ | 0 | 100 |

(CFU Denotes Colony Counting Units)

Referring to Table 4 above, it was confirmed that, in the case in which the antimicrobial pad was not coated with the antimicrobial composition or was coated with the antimicrobial composition obtained by mixing the extracts in amounts less than the lower limits of the ranges specified in the present disclosure, the concentration of the fungus increased.

On the other hand, it was confirmed that, when the antimicrobial composition of the present disclosure was used, the concentration of the fungus decreased, suggesting that the antimicrobial composition has an excellent antimicrobial effect.

### EXPERIMENTAL EXAMPLE 4

Sensory Evaluation

To perform the sensory evaluation of the sanitary napkin according to an embodiment of the present disclosure, 15 women in their 20s were divided into five groups, each consisting of 3 women, and the sensory evaluation was performed on the five groups.

Wearing sensation, the degree of absorption, the occurrence of odor, and whether menstrual blood leaks were examined. These items were evaluated in in a five-point scale, and the higher the evaluation score, the better the evaluation.

The sanitary napkins prepared to have AR1 to AR4 and the comparative example as antimicrobial pads, respectively, were provided, and layers other than the antimicrobial pad were all the same among the sanitary napkins.

TABLE 5

| | | | | | Comparative |
|---|---|---|---|---|---|
| | AR1 | AR2 | AR3 | AR4 | Example |
| Wearing | 5 | 4.5 | 4.5 | 5 | 4.5 |
| sensation | | | | | |
| Degree of | 4.5 | 5 | 4.5 | 5 | 4.5 |
| absorption | | | | | |
| Odor | 2 | 4 | 4.5 | 5 | 1 |
| Whether | 5 | 5 | 5 | 5 | 5 |
| menstrual | | | | | |
| blood leaked | | | | | |

According to the above evaluation results, it was confirmed that the wearing sensation, the degree of absorption and whether menstrual blood leaked all received excellent evaluation. In addition, the subjects were asked whether they would like to purchase the sanitary napkins when the sanitary napkins would be sold as actual products. As a result, it was confirmed that the subjects of the AR2 to AR4 groups had high intention to purchase if the sanitary napkins would not significantly high in price. However, it was confirmed that the subjects of the AR1 group and the comparative example group easily felt the odor of menstrual blood and gave very low evaluation on the odor, and had low intention to purchase the sanitary napkins.

Although the preferred embodiments of the present disclosure have been described above in detail, the scope of the present disclosure is not limited thereto. Those skilled in the art will appreciate that various modifications and improvements are possible, without departing from the basic concept of the present disclosure as defined in the appended claims, and also fall within the scope of the present disclosure.

### INDUSTRIAL APPLICABILITY

The disclosure relates to a water-degradable environmentally friendly sanitary napkin, and more particularly to a water-degradable environmentally friendly sanitary napkin whose constituent components are degradable in a large amount of water or biodegradable in soil, thus preventing environmental pollution problems.

1. A water-degradable environmentally friendly sanitary napkin comprising:
a permeable inner sheet configured to come into contact with a skin and allow menstrual blood to pass therethrough;
an impermeable waterproof outer sheet having the same size and shape as those of the inner sheet and bonded to the inner sheet by fusion;
an absorbent pad fixedly inserted between the inner sheet and the waterproof outer sheet and configured to absorb the menstrual blood that passes through the inner sheet; and
an adhesive pad fixed to a lower surface of the waterproof outer sheet and configured to fix the sanitary napkin by adhesion to an inside of underwear,
wherein the inner sheet, the waterproof outer sheet, the absorbent pad and the adhesive pad disintegrate water or biodegrade.

2. The water-degradable environmentally friendly sanitary napkin of claim 1, wherein
one surface of the waterproof outer sheet is in contact with the absorbent pad, the adhesive pad is fixed to the other surface of the waterproof outer sheet,
the one surface of the waterproof outer sheet, which is in contact with the absorbent pad, prevents menstrual blood, absorbed by the absorbent pad, from passing therethrough, due to the water-repellent effect thereof, and
the other surface of the waterproof outer sheet, to which the adhesive pad is fixed, easily absorbs water, and thus disintegrates in water.

3. The water-degradable environmentally friendly sanitary napkin of claim 1, wherein
the waterproof outer sheet is composed of pulp, and
one surface of the waterproof outer sheet exhibits a water-repellent effect due to bonding between a hydroxyl group (—OH) of the pulp and a compound represented by the following Formula 1 or Formula 2: 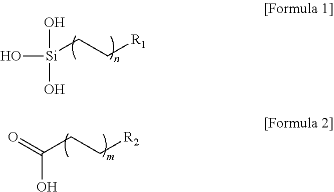
wherein
n and m are the same or different and are each independently an integer ranging from 1 to 10;
R₁ and R₂ are the same or different and are each independently a substituted or unsubstituted C₁-C₁₀ alkyl group;
R₃ is a halogen group; and
R₁ and R₂ are optionally substituted with a halogen group.

4. The water-degradable environmentally friendly sanitary napkin of claim 1, wherein the absorbent pad is composed of a plurality of absorbent layers, and the absorbent layers are made of a pulp material.

5. The water-degradable environmentally friendly sanitary napkin of claim 1, wherein wing portions are formed at both lateral sides of the adhesive pad, and an adhesive layer for adhesion to the inside of underwear is formed on one surface of each of the wing portions.

6. The water-degradable environmentally friendly sanitary napkin of claim 5, wherein the adhesive layer comprises a hydrocarbon resin.

7. The water-degradable environmentally friendly sanitary napkin of claim 1, further comprising an antimicrobial pad disposed between the inner sheet and the absorbent pad and configured to remove odor of menstrual blood before the menstrual blood that passes through the inner sheet is absorbed into the absorbent pad.
